SEQUENCE LISTING (50 sequences)

SEQ ID NO: 1 - DNA, 20 bp, Artificial Sequence
  Synthetic oligonucleotide primer
atgcttccttttcgtcattc

SEQ ID NO: 2 - DNA, 20 bp, Artificial Sequence
  Synthetic oligonucleotide primer
caaacaacatgagccagaag

SEQ ID NO: 3 - DNA, 22 bp, Artificial Sequence
  Synthetic oligonucleotide primer
ttcttaaaagtagctgtccctc

SEQ ID NO: 4 - DNA, 19 bp, Artificial Sequence
  Synthetic oligonucleotide primer
ctcgagcatggtcagaaag

SEQ ID NO: 5 - DNA, 17 bp, Artificial Sequence
  Synthetic oligonucleotide primer
acctgtccctccaccag

SEQ ID NO: 6 - DNA, 21 bp, Artificial Sequence
  Synthetic oligonucleotide primer
ctaccagacacccctaaaatg

SEQ ID NO: 7 - DNA, 20 bp, Artificial Sequence
  Synthetic oligonucleotide primer
atggaagcagataaaccagg

SEQ ID NO: 8 - DNA, 18 bp, Artificial Sequence
  Synthetic oligonucleotide primer
agaagagaaagggccgtc

SEQ ID NO: 9 - DNA, 19 bp, Artificial Sequence
  Synthetic oligonucleotide primer
gaggaccagagactccagg

SEQ ID NO: 10 - DNA, 17 bp, Artificial Sequence
  Synthetic oligonucleotide primer
gagcctgttcctcaggg

SEQ ID NO: 11 - DNA, 19 bp, Artificial Sequence
  Synthetic oligonucleotide primer
agagggaaacgccactaac

SEQ ID NO: 12 - DNA, 22 bp, Artificial Sequence
  Synthetic oligonucleotide primer
ttcactaccattattttgcagc

SEQ ID NO: 13 - DNA, 19 bp, Artificial Sequence
  Synthetic oligonucleotide primer
gcttgttgctgttttgttc

SEQ ID NO: 14 - DNA, 19 bp, Artificial Sequence
  Synthetic oligonucleotide primer
gtctccactttcatgcagc

SEQ ID NO: 15 - DNA, 19 bp, Artificial Sequence
  Synthetic oligonucleotide primer
gcatctttctcctgctctg

SEQ ID NO: 16 - DNA, 18 bp, Artificial Sequence
  Synthetic oligonucleotide primer
gctgccctgaactacctg

SEQ ID NO: 17 - DNA, 20 bp, Artificial Sequence
  Synthetic oligonucleotide primer
cagttgcacctcacttttcc

SEQ ID NO: 18 - DNA, 19 bp, Artificial Sequence
  Synthetic oligonucleotide primer
ctctcattaggcagggagg

SEQ ID NO: 19 - DNA, 18 bp, Artificial Sequence
  Synthetic oligonucleotide primer
tgggaatgctcctgaaac

SEQ ID NO: 20 - DNA, 20 bp, Artificial Sequence
  Synthetic oligonucleotide primer
caattgagaggcaggatttc

SEQ ID NO: 21 - DNA, 24 bp, Artificial Sequence
  Synthetic oligonucleotide primer
ttcttgtgtgtacaaggttatctg

SEQ ID NO: 22 - DNA, 20 bp, Artificial Sequence
  Synthetic oligonucleotide primer
cacctaaggagtcttctggc

SEQ ID NO: 23 - DNA, 19 bp, Artificial Sequence
  Synthetic oligonucleotide primer
cggtgaacagaggtttcag

SEQ ID NO: 24 - DNA, 18 bp, Artificial Sequence
  Synthetic oligonucleotide primer
gggaggacagaggtgagg

SEQ ID NO: 25 - DNA, 19 bp, Artificial Sequence
  Synthetic oligonucleotide primer
tgtgtcagtcccctagcac

SEQ ID NO: 26 - DNA, 21 bp, Artificial Sequence
  Synthetic oligonucleotide primer
gccgagaagtaactgaacatc

SEQ ID NO: 27 - DNA, 19 bp, Artificial Sequence
  Synthetic oligonucleotide primer
gtgggagggagactggtag

SEQ ID NO: 28 - DNA, 22 bp, Artificial Sequence
  Synthetic oligonucleotide primer
gatcttaagtctgtcaaatggc

SEQ ID NO: 29 - DNA, 20 bp, Artificial Sequence
  Synthetic oligonucleotide primer
tgtgtagcaaaatggcactc

SEQ ID NO: 30 - DNA, 20 bp, Artificial Sequence
  Synthetic oligonucleotide primer
gggctaactcacatttcagc

SEQ ID NO: 31 - DNA, 20 bp, Artificial Sequence
  Synthetic oligonucleotide primer
taaatccagaggacgaggtc

SEQ ID NO: 32 - DNA, 19 bp, Artificial Sequence
  Synthetic oligonucleotide primer
gaaaggaaagagccgtgac

SEQ ID NO: 33 - DNA, 19 bp, Artificial Sequence
  Synthetic oligonucleotide primer
tgatgactacttgccccag

SEQ ID NO: 34 - DNA, 17 bp, Artificial Sequence
  Synthetic oligonucleotide primer
tcagtgcaggctcaggc

SEQ ID NO: 35 - DNA, 20 bp, Artificial Sequence
  Synthetic oligonucleotide primer
acagtgtgtttgagacgtgg

SEQ ID NO: 36 - DNA, 17 bp, Artificial Sequence
  Synthetic oligonucleotide primer
aagaggcctcacaagcc

SEQ ID NO: 37 - DNA, 17 bp, Artificial Sequence
  Synthetic oligonucleotide primer
tgatgcgtttcaccgac

SEQ ID NO: 38 - DNA, 18 bp, Artificial Sequence
  Synthetic oligonucleotide primer
gaaagggagccgtctcag

SEQ ID NO: 39 - DNA, 20 bp, Artificial Sequence
  Synthetic oligonucleotide primer
ggtgacatgcactctaaggg

SEQ ID NO: 40 - DNA, 20 bp, Artificial Sequence
  Synthetic oligonucleotide primer
ttccacttgaagctctcctc

SEQ ID NO: 41 - DNA, 20 bp, Artificial Sequence
  Synthetic oligonucleotide primer
attccagaatctcagcggac

SEQ ID NO: 42 - DNA, 18 bp, Artificial Sequence
  Synthetic oligonucleotide primer
ctctggggacaggagacc

SEQ ID NO: 43 - DNA, 17 bp, Artificial Sequence
  Synthetic oligonucleotide primer
ttcctctgaagcggtgg

SEQ ID NO: 44 - DNA, 17 bp, Artificial Sequence
  Synthetic oligonucleotide primer
cgcagcctaactgcctg

SEQ ID NO: 45 - DNA, 18 bp, Artificial Sequence
  Synthetic oligonucleotide primer
tgtgagagctcctgctcg

SEQ ID NO: 46 - DNA, 18 bp, Artificial Sequence
  Synthetic oligonucleotide primer
cggccctgtagtcacatc

SEQ ID NO: 47 - DNA, 19 bp, Artificial Sequence
  Synthetic oligonucleotide primer
aggccaagagtctcctctg

SEQ ID NO: 48 - DNA, 18 bp, Artificial Sequence
  Synthetic oligonucleotide primer
ttcgtttcaggagccttg

SEQ ID NO: 49 - DNA, 17 bp, Artificial Sequence
  Synthetic oligonucleotide primer
gtcgtgcctggttgttc

SEQ ID NO: 50 - DNA, 17 bp, Artificial Sequence
  Synthetic oligonucleotide primer
aaaatgctggctttgcc

(19) United States
(12) Patent Application Publication
(10) Pub. No.: US 2015/0106960 A1
(43) Pub. Date: Apr. 16, 2015

(54) METHOD FOR DIAGNOSING A SKELETAL CILIOPATHY

(71) Applicants: INSERM (INSTITUT NATIONAL DE LA SANTE ET DE LA RECHERCHE MEDICALE), Paris (FR); ASSISTANCE PUBLIQUE HOPITAUX DE PARIS, Paris (FR); UNIVERSITE PARIS DESCARTES, Paris (FR); FONDATION IMAGINE, Paris (FR)

(72) Inventors: Valerie Cormier-Daire, Paris (FR); Josseline Kaplan, Paris (FR); Isabelle Perrault, Paris (FR); Jean-Michel Rozet, Paris (FR); Arnold Munnich, Paris (FR); Sophie Saunier, Paris (FR)

(21) Appl. No.: 14/391,172

(22) PCT Filed: Apr. 10, 2013
(86) PCT No.: PCT/EP2013/057438

§ 371 Date: Oct. 8, 2014

(30) Foreign Application Priority Data
Apr. 11, 2012 (EP) 12305428.0

(51) Int. Cl. C12Q 1/68; A01K 67/027

(52) U.S. Cl. CPC C12Q 1/6883; A01K 67/0275; C12Q 2600/156; A01K 2267/0306
USPC 800/9; 435/6.12; 435/6.11; 514/44 R

(57) ABSTRACT

The present invention relates to a method for diagnosing a skeletal ciliopathy.

### FIELD OF THE INVENTION

The present invention relates to a method for diagnosing a skeletal ciliopathy.

### BACKGROUND OF THE INVENTION

Ciliopathies comprise a group of disorders associated with genetic mutations encoding defective proteins, which result in either abnormal formation or function of cilia. As cilia are a component of almost all vertebrate cells, cilia dysfunction can manifest as a constellation of features that include characteristically, retinal degeneration, renal disease and cerebral anomalies. Additional manifestations include congenital fibrocystic diseases of the liver, diabetes, obesity and skeletal dysplasias. Ciliopathic features have been associated with mutations in over 40 genes to date. However, with over 1,000 polypeptides currently identified within the ciliary proteome, several other disorders associated with this constellation of clinical features will likely be ascribed to mutations in other ciliary genes. The mechanisms underlying many of the disease phenotypes associated with ciliary dysfunction have yet to be fully elucidated².

At present, there is no method for early diagnosing some ciliopathies, such as Mainzer Saldino syndrome. Then, these pathologies are diagnosed at a late stage whereas an early diagnostic would allow stopping or slowing the evolution of the main serious manifestations of the disease.

### SUMMARY OF THE INVENTION

The present invention relates to a method for diagnosing a skeletal ciliopathy in a subject, said method comprising detecting a mutation in the IFT140 gene in a biological sample obtained from said subject, wherein the presence of an IFT140 mutation is indicative of a skeletal ciliopathy.

The present invention also relates to a method for predicting a risk of a subject to transmit a skeletal ciliopathy to his enfants, said method comprising detecting a mutation in the IFT140 gene in a biological sample obtained from said subject, wherein the presence of an IFT140 mutation is indicative of a risk of transmitting a skeletal ciliopathy.

The present invention relates to a method for detecting a subject carrying a defective IFT140 gene, which method comprises detecting a mutation in the IFT140 gene in a biological sample obtained from said subject.

The present invention also relates to an IFT140-encoding polynucleotide for use in therapy.

The present invention also relates to an IFT140-encoding polynucleotide for treating a skeletal ciliopathy.

The present invention also relates to a transgenic non-human animal which is IFT140-deficient.

### DETAILED DESCRIPTION OF THE INVENTION

### Definitions

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

As used herein, the term “IFT140 gene” denotes the IFT140 gene of any species, especially human, but also other mammals or vertebrates to which the methods of the invention can apply.

The human IFT140 gene located at 16p13.3, covers a genomic region of 101682 bp and is composed of 31 exons (29 coding). The transcript is 5277 bp long, with a coding sequence of 4389 bp. The encoded IFT140 protein (intraflagellar transport protein 140 homolog) is 1462 amino-acids long (NP_{—}055529.2).

We have chosen to number the A of the start codon (ATG) of the cDNA sequence of IFT140 (NCBI Reference Sequence: NM_{—}014714.3) as nucleotide 1.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989).

The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tm, e.g., 40% formamide, with 5× or 6×SCC. High stringency hybridization conditions correspond to the highest Tm, e.g., 50% formamide, 5× or 6×SCC. SCC is a 0.15 M NaCl, 0.015 M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., 1989, 9.50-9.51). For hybridization with shorter nucleic acids, i.e., oligonucleotides, the position of mismatches becomes more important, and the length of the oligonucleotide determines its specificity (see Sambrook et al., 1989 11.7-11.8). A minimum length for a hybridizable nucleic acid is at least about 10 nucleotides, preferably at least about 15 nucleotides, and more preferably the length is at least about 20 nucleotides.

In a specific embodiment, the term “standard hybridization conditions” refers to a Tm of 55° C., and utilizes conditions as set forth above. In a preferred embodiment, the Tm is 60° C. In a more preferred embodiment, the Tm is 65° C. In a specific embodiment, “high stringency” refers to hybridization and/or washing conditions at 68° C. in 0.2×SSC, at 42° C. in 50 formamide, 4×SSC, or under conditions that afford levels of hybridization equivalent to those observed under either of these two conditions.

As used herein, an amplification primer is an oligonucleotide for amplification of a target sequence by extension of the oligonucleotide after hybridization to the target sequence or by ligation of multiple oligonucleotides which are adjacent when hybridized to the target sequence. At least a portion of the amplification primer hybridizes to the target. This portion is referred to as the target binding sequence and it determines the target-specificity of the primer. In addition to the target binding sequence, certain amplification methods require specialized non-target binding sequences in the amplification primer. These specialized sequences are necessary for the amplification reaction to proceed and typically serve to append the specialized sequence to the target. For example, the amplification primers used in Strand Displacement Amplification (SDA) include a restriction endonuclease recognition site 5′ to the target binding sequence (U.S. Pat. No. 5,455,166 and U.S. Pat. No. 5,270,184). Nucleic Acid Based Amplification (NASBA), self-sustaining sequence replication (3SR) and transcription based amplification primers require an RNA polymerase promoter linked to the target binding sequence of the primer. Linking such specialized sequences to a target binding sequence for use in a selected amplification reaction is routine in the art. In contrast, amplification methods such as PCR which do not require specialized sequences at the ends of the target, generally employ amplification primers consisting of only target binding sequence.

As used herein, the terms “primer” and “probe” refer to the function of the oligonucleotide. A primer is typically extended by polymerase or ligation following hybridization to the target but a probe typically is not. A hybridized oligonucleotide may function as a probe if it is used to capture or detect a target sequence, and the same oligonucleotide may function as a primer when it is employed as a target binding sequence in an amplification primer. It will therefore be appreciated that any of the target binding sequences disclosed herein for amplification, detection or quantisation of IFT140 may be used either as hybridization probes or as target binding sequences in primers for detection or amplification, optionally linked to a specialized sequence required by the selected amplification reaction or to facilitate detection.

The terms “mutant” and “mutation” mean any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. Generally a mutation is identified in a subject by comparing the sequence of a nucleic acid or polypeptide expressed by said subject with the corresponding nucleic acid or polypeptide expressed in a control population. A mutation in the genetic material may also be “silent”, i.e. the mutation does not result in an alteration of the amino acid sequence of the expression product.

In the context of the instant application, mutations identified in IFT140 gene are designated pursuant to the nomenclature of Dunnen and Antonarakis (2000).

As defined by Dunnen and Antonarakis, to avoid confusion in the description of a sequence change, preceed the description with a letter indicating the type of reference sequence used:“g.” for a genomic sequence (e.g., g.76A>T), “c.” for a cDNA sequence (e.g., c.76A>T), “m.” for a mitochondrial sequence (e.g., m.76A>T), “r.” for an RNA sequence (e.g., r.76a>u), “p.” for a protein sequence (e.g., p.K76A).

Substitutions are designated by a “>”-character:
76A>C denotes that at nucleotide 76 an A is changed to a C
88+1G>T denotes the G to T substitution at nucleotide+1 of intron 2, relative to the cDNA positioned between nucleotides 88 and 89

Deletions are designated by “del” after the nucleotide(s) flanking the deletion site:
76_{—}78del (alternatively 76_{—}78delACT) denotes a ACT deletion from nucleotides 76 to 78

Duplications are designated by “dup” after the first and last nucleotide affected by the duplication:
77-79dup (or 77_{—}79dupCTG) denotes that the nucleotides 77 to 79 were duplicated

Thus, for example, “c.1990G>A” denotes that at nucleotide 1990 of the cDNA sequence a G is changed to a A.

The expression “homozygous IFT140 mutation”, as used herein, refers to a subject whose two alleles of the IFT140 gene are mutated. The expression thus encompasses both homozygous mutations strico sensu (wherein the same mutation is present on both alleles) and compound heterozygous mutation (wherein each allele presents a different mutation).

In the context of the invention, the term “treating” or “treatment”, as used herein, means reversing, alleviating, inhibiting the progress of, or preventing the disorder or condition to which such term applies, or one or more symptoms of such disorder or condition. A “therapeutically effective amount” is intended for a minimal amount of active agent (e.g., IFT140 encoding polynucleotide) which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with or resistance to succumbing to a disorder.

Mainzer-Saldino syndrome (MSS, [MIM 266920]) or conorenal syndrome (CRS) is a rare autosomal recessive disease defined by phalangeal cone-shaped epiphyses (POSE), chronic renal disease, nearly constant retinal dystrophy and mild radiographic abnormality of the proximal femur¹². Occasional features include short stature, cerebellar ataxia and hepatic fibrosis¹². MSS shares retinal dystrophy with Leber congenital amaurosis (LCA [MIM 204000]), nephronophthisis (NPHP [MIM 256100]) and skeletal features with asphyxiating thoracic dystrophy or Jeune syndrome (ATD [MIM 208500]) and cranioectodermal dysplasia or Sensenbrenner syndrome (CED [MIM 218330]).

### Diagnostic Methods of the Invention

The present invention relates to method for diagnosing a skeletal ciliopathy in a subject, said method comprising detecting a mutation in the IFT140 gene in a biological sample obtained from said subject, wherein the presence of an IFT140 mutation is indicative of a skeletal ciliopathy or a risk of having a skeletal ciliopathy.

Indeed, the inventors have shown that mutations found in the IFT140 gene are associated with a skeletal ciliopathy. Defective IFT140 is involved in the pathological process of skeletal ciliopathy.

The terms “biological sample” means any biological sample derived from a subject. Examples of such samples include fluids, tissues, cell samples, tissue biopsies, etc.

Preferred biological samples are a cell or tissue sample.

Preferred biological samples are whole blood, serum or plasma.

Typically, in the case where the subject is a foetus, the sample may be an amniocentesis sample.

Typically, the subject according to the invention is a human.

The subject may be an adult, a teenager, a child, an infant, a baby or a foetus.

The method of the invention may be used a prenatal method for diagnosing or predicting a skeletal ciliopathy.

In a preferred embodiment, the subject has a retinal dystrophy.

Indeed, retinal dystrophy is one of the first symptoms of some skeletal ciliopathies such as Mainzer Saldino Syndrome (MSS).

By testing a subject afflicted with a retinal dystrophy according to the method of the invention, a skeletal ciliopathy may be diagnosed early and then the subject may be treated to slow the evolution of the disease.

For example, in MSS, the retinal dystrophy appears early and the MSS could lead to chronic renal disease whose evolution could be slowed or stopped by an early diagnosis of MSS.

The present invention also relates to a method for predicting a risk of a subject to transmit a skeletal ciliopathy to his infants, said method comprising detecting a mutation in the IFT140 gene in a biological sample obtained from said subject, wherein the presence of an IFT140 mutation is indicative of a risk of transmitting a skeletal ciliopathy.

In a preferred embodiment, the skeletal ciliopathy is a Mainzer Saldino Syndrome or a Jeune Syndrome.

In the most preferred embodiment, the skeletal ciliopathy is a Mainzer Saldino Syndrome.

The present invention also relates to method for detecting a subject carrying a defective IFT140 gene, which method comprises detecting a mutation in the IFT140 gene in a biological sample obtained from said subject.

An IFT140 mutation according to the invention may be found in a regulating region of IFT140 gene (e.g. a promoter sequence, or a binding site for transcription factor), in introns or in exons that encode the IFT140 protein.

Typically, the IFT140 mutation is a mutation which results in IFT140 defective gene.

An IFT140 defective gene results in a defect in ciliogenesis and/or cilia maintenance.

In an embodiment, the IFT140 mutation is a mutation which results in a truncated IFT140 protein, an IFT140 mislocalization or in a reduction of IFT140 expression.

The IFT140 mutation may result in an IFT140 mislocalization that is to say aberrant localization compared to controls carrying no compound or homozygote IFT140 mutations.

The IFT140 mutation may also result in a normal IFT140 localization but abnormal localization of other IFT proteins that is to say aberrant localization compared to controls carrying no compound or homozygote IFT140 mutations.

The IFT140 mutation may also results in an alteration in retrograde ciliary transport.

Typically, the IFT140 mutation is a missense mutation, a donor splice mutation or a truncating mutation.

Preferably, the IFT140 mutation is an IFT140 mutation is selected from the group consisting of c.2399+1G>T, c.932A>G, c.1990G>A, c.634G>A, c.699T>G, c.1565G>A, c.874G>A, c.1727G>A, c.489C>T, c.857_{—}860deITTGA and c.3916dup.

In an embodiment, the IFT140 mutation is heterozygous.

In an embodiment, the IFT140 mutation is heterozygous and selected from the group consisting of c.1565G>A, c.874G>A, c.1727G>A and c.489C>T.

In a more preferred embodiment, the IFT140 mutation is homozygous.

In a more preferred embodiment, the IFT140 mutation is homozygous and selected from the group consisting of c.2399+1G>T, c.932A>G, c.1990G>A, c.634G>A, c.699T>G, c.857_{—}860deITTGA and c.3916dup.

In a preferred embodiment, the mutation is a homozygous mutation stricto sensu, wherein both alleles of the IFT140 gene present a c.1990G>A or c.699T>G substitution.

In another embodiment, the mutation is a composite heterozygous mutation, wherein one allele of the IFT140 gene presents an IFT140 mutation is selected from the group consisting of c.2399+1G>T, c.932A>G, c.1990G>A, c.634G>A, c.699T>G, c.857_{—}860deITTGA, and c.3916dup.

IFT140 mutations may be detected by analyzing a IFT140 nucleic acid molecule. In the context of the invention, IFT140 nucleic acid molecules include mRNA, genomic DNA and cDNA derived from mRNA. DNA or RNA can be single stranded or double stranded.

DNA may be extracted using any methods known in the art, such as described in Sambrook et al., 1989.

RNA may also be isolated, for instance from tissue biopsy, using standard methods well known to the one skilled in the art such as guanidium thiocyanate-phenol-chloroform extraction.

IFT140 mutations may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of a particular IFT140 mutation. Otherwise, RNA may be reverse-transcribed and amplified, or DNA may be amplified, after which a mutated site may be detected in the amplified sequence by hybridization with a suitable probe or by direct sequencing, or any other appropriate method known in the art. For instance, a cDNA obtained from RNA may be cloned and sequenced to identify a mutation in IFT140 sequence. Actually numerous strategies for genotype analysis are available (Antonarakis et al., 1989; Cooper et al., 1991; Grompe, 1993). Briefly, the nucleic acid molecule may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized.

Therefore, useful nucleic acid molecules, in particular oligonucleotide probes or primers, according to the present invention include those which specifically hybridize the regions where the mutations are located.

Oligonucleotide probes or primers may contain at least 10, 15, 20 or 30 nucleotides. Their length may be shorter than 400, 300, 200 or 100 nucleotides.

According to a further embodiment said mutation in the IFT140 gene may be detected at the protein level.

Said mutation may be detected according to any appropriate method known in the art. In particular a biological sample obtained from a subject may be contacted with antibodies specific of the mutated form of IFT140, i.e. antibodies that are capable of distinguishing between a mutated form of IFT140 and the wild-type protein (or any other protein), to determine the presence or absence of a IFT140 specified by the antibody.

Antibodies that specifically recognize a mutated IFT140 also make part of the invention. The antibodies are specific of mutated IFT140, that is to say they do not cross-react with the wild-type IFT140.

The antibodies of the present invention may be monoclonal or polyclonal antibodies, single chain or double chain, chimeric antibodies, humanized antibodies, or portions of an immunoglobulin molecule, including those portions known in the art as antigen binding fragments Fab, Fab′, F(ab′)2 and F(v). They can also be immunoconjugated, e.g. with a toxin, or labelled antibodies.

Whereas polyclonal antibodies may be used, monoclonal antibodies are preferred for they are more reproducible in the long run.

Procedures for raising “polyclonal antibodies” are also well known. Polyclonal antibodies can be obtained from serum of an animal immunized against the appropriate antigen, which may be produced by genetic engineering for example according to standard methods well-known by one skilled in the art. Typically, such antibodies can be raised by administering mutated IFT140 subcutaneously to New Zealand white rabbits which have first been bled to obtain pre-immune serum. The antigens can be injected at a total volume of 100 μl per site at six different sites. Each injected material may contain adjuvants with or without pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. This and other procedures for raising polyclonal antibodies are disclosed by Harlow et al. (1988) which is hereby incorporated in the references.

A “monoclonal antibody” in its various grammatical forms refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the present invention.

Laboratory methods for preparing monoclonal antibodies are well known in the art (see, for example, Harlow et al., 1988). Monoclonal antibodies (mAbs) may be prepared by immunizing purified mutated IFT140 into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

While mAbs can be produced by hybridoma culture the invention is not to be so limited. Also contemplated is the use of mAbs produced by an expressing nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; PCT Publication No.; European Patent Publications No. 0239400 to Winter et al. and No. 0125023 to Cabilly et al.

Antibody generation techniques not involving immunisation are also contemplated such as for example using phage display technology to examine naive libraries (from non-immunised animals); see Barbas et al. (1992), and Waterhouse et al. (1993).

Antibodies raised against mutated IFT140 may be cross reactive with wild-type IFT140. Accordingly a selection of antibodies specific for mutated IFT140 is required. This may be achieved by depleting the pool of antibodies from those that are reactive with the wild-type IFT140, for instance by submitting the raised antibodies to an affinity chromatography against wild-type IFT140.

Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Probe, primers, aptamers or antibodies of the invention may be labelled with a detectable molecule or substance, such as a fluorescent molecule, a radioactive molecule or any others labels known in the art. Labels are known in the art that generally provide (either directly or indirectly) a signal.

The term “labelled”, with regard to the probe, primers, aptamers or antibodies of the invention, is intended to encompass direct labelling of the the probe, primers, aptamers or antibodies of the invention by coupling (i.e., physically linking) a detectable substance to the the probe, primers, aptamers or antibodies of the invention, as well as indirect labeling of the probe, primers, aptamers or antibodies of the invention by reactivity with another reagent that is directly labeled. Other examples of detectable substances include but are not limited to radioactive agents or a fluorophore (e.g. fluorescein isothiocyanate (FITC) or phycoerythrin (PE) or Indocyanine (Cy5)). Examples of indirect labeling include detection of a primary antibody using a fluorescently labeled secondary antibody and end-labeling of a DNA probe with biotin such that it can be detected with fluorescently labeled streptavidin. An antibody or aptamer of the invention may be labelled with a radioactive molecule by any method known in the art. For example radioactive molecules include but are not limited radioactive atom for scintigraphic studies such as I123, I124, In111, Re186, Re188.

### Kits of the Invention

According to another aspect of the invention, the IFT140 mutation is detected by contacting the DNA of the subject with a nucleic acid probe, which is optionally labeled.

Primers may also be useful to amplify or sequence the portion of the IFT140 gene containing the mutated positions of interest.

Such probes or primers are nucleic acids that are capable of specifically hybridizing with a portion of the IFT140 gene sequence containing the mutated positions of interest. That means that they are sequences that hybridize with the portion mutated IFT140 nucleic acid sequence to which they relate under conditions of high stringency.

The present invention further provides kits suitable for determining at least one of the mutations of the IFT140 gene.

The kits may include the following components:
(i) a probe, usually made of DNA, and that may be pre-labelled. Alternatively, the probe may be unlabelled and the ingredients for labelling may be included in the kit in separate containers; and
(ii) hybridization reagents: the kit may also contain other suitably packaged reagents and materials needed for the particular hybridization protocol, including solid-phase matrices, if applicable, and standards.

In another embodiment, the kits may include:
(i) sequence determination or amplification primers: sequencing primers may be pre-labelled or may contain an affinity purification or attachment moiety; and
(ii) sequence determination or amplification reagents: the kit may also contain other suitably packaged reagents and materials needed for the particular sequencing amplification protocol. In one preferred embodiment, the kit comprises a panel of sequencing or amplification primers, whose sequences correspond to sequences adjacent to at least one of the polymorphic positions, as well as a means for detecting the presence of each polymorphic sequence.

In a particular embodiment, it is provided a kit which comprises a pair of nucleotide primers specific for amplifying all or part of the IFT140 gene comprising at least one of mutations that are identified herein, especially IFT140 mutations selected from the group consisting of c.2399+1G>T, c.932A>G, c.1990G>A, c.634G>A, c.699T>G, c.1565G>A, c.874G>A, c.1727G>A, c.489C>T, c.857_{—}860deITTGA and c.3916dup.

Alternatively, the kit of the invention may comprise a labelled compound or agent capable of detecting the mutated polypeptide of the invention (e.g., an antibody or aptamers as described above which binds the polypeptide). For example, the kit may comprise (1) a first antibody (e.g., attached to a solid support) which binds to a polypeptide comprising a mutation of the invention; and, optionally, (2) a second, different antibody which binds to either the polypeptide or the first antibody and is conjugated to a detectable agent.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a skeletal ciliopathy.

### Therapeutic Methods of the Invention

In a further object, the invention relates to use, methods and pharmaceutical compositions for treating a skeletal ciliopathy, and more particularly Mainzer Saldino Syndrome. Gene therapy is a particularly convenient way to treat a skeletal ciliopathy as it enables the provision of a constant supply of polypeptide or correction of the defective gene, for example as discussed below.

Gene therapy may be carried out by means of supplementation of cells lacking a functional IFT140 polypeptide with a wild type IFT140 gene product. Production of a suitable gene product may be achieved using recombinant techniques. For example, a suitable vector may be inserted into a host cell and expressed in that cell.

Thus the invention further relates to a method for treating a skeletal ciliopathy which comprises the step of administering a subject in need thereof with an IFT140 polynucleotide, i.e. a nucleic acid sequence that encodes a wild-type IFT140, so that IFT140 is expressed in vivo by the cells of the subject that have been transfected with said polynucleotide. Accordingly, said method leads to an overexpression of wild-type IFT140 which compensates expression of defective mutated IFT140.

The invention relates to an IFT140-encoding polynucleotide for use in therapy. The IFT140-encoding polynucleotide may be for use in a method of treatment of a skeletal ciliopathy and more particularly a Mainzer Saldino Syndrome.

The invention also relates to the use of a IFT140 polynucleotide for the manufacture of medicament intended for the treatment of a skeletal ciliopathy.

Said IFT140 polynucleotide is administered in a therapeutically effective amount.

Preferably the IFT140 polynucleotide sequence according to the invention is associated with elements that enable for regulation of its expression, such as a promoter sequence.

Such a nucleic acid may be in the form of a vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses (AAV)), which serve equivalent functions.

In a preferred embodiment, the expression vector is an AAV vector.

Adeno-associated viral (AAV) vectors have become powerful gene delivery tools for the treatment of retinal degeneration. AAV vectors possess a number of features that render them ideally suited for retinal gene therapy, including a lack of pathogenicity, minimal immunogenicity, and the ability to transduce postmitotic cells in a stable and efficient manner. In the sheltered environment of the retina, AAV vectors are able to maintain high levels of transgene expression in the retinal pigmented epithelium (RPE), photoreceptors, or ganglion cells for long periods of time after a single treatment. Each cell type can be specifically targeted by choosing the appropriate combination of AAV serotype, promoter, and intraocular injection site (Dinculescu et al., Hum Gene Ther. 2005 June; 16(6):649-63).

The IFT140 polynucleotide may be introduced into a target cell by means of any procedure known for the delivery of nucleic acids to the nucleus of cells, ex vivo, on cells in culture or removed from an animal or a patient, or in vivo.

Ex vivo introduction may be performed by any standard method well known by one skilled in the art, e.g. transfection, electroporation, microinjection, transduction, cell fusion, DEAE dextran, calcium phosphate precipitation, or use of a gene gun.

The IFT140 polynucleotide can also be introduced ex vivo or in vivo by lipofection. In certain embodiments, the use of liposomes and/or nanoparticles is contemplated for the introduction of the donor nucleic acid targeting system into host cells.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core.

Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes (Feigner et al., 1989).

Alternatively, one of the simplest and the safest way to deliver IFT140 polynucleotide across cell membranes in vivo may involve the direct application of high concentration free or naked polynucleotides (typically mRNA or DNA). By “naked DNA (or RNA)” is meant a DNA (RNA) molecule which has not been previously complexed with other chemical moieties. Naked DNA uptake by animal cells may be increased by administering the cells simultaneously with excipients and the nucleic acid. Such excipients are reagents that enhance or increase penetration of the DNA across cellular membranes and thus delivery to the cells delivery of the therapeutic agent. Various excipients have been described in the art, such as surfactants, e.g. a surfactant selected form the group consisting of Triton X-100, sodium dodecyl sulfate, Tween 20, and Tween 80; bacterial toxins, for instance streptolysin O, cholera toxin, and recombinant modified labile toxin of E. coli; and polysaccharides, such as glucose, sucrose, fructose, or maltose, for instance, which act by disrupting the osmotic pressure in the vicinity of the cell membrane. Other methods have been described to enhance delivery of free polynucleotides, such as blocking of polynucleotide inactivation via endo- or exonucleolytic cleavage by both extra- and intracellular nucleases.

Alternatively, the invention also provides a method for treating a skeletal ciliopathy which comprises the step of administering a subject in need thereof with a wild-type IFT140.

Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

Alternatively, the polypeptides of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

Polypeptides of the invention can be use in an isolated (e.g., purified) form or contained in a vector, such as a membrane or lipid vesicle (e.g. a liposome).

### Transgenic Non-Human Animal Models of Skeletal Ciliopathy

The present invention further relates to an in vivo model of skeletal ciliopathy and more particularly of Mainzer Saldino Syndrome: a transgenic non-human animal which is IFT140-deficient.

By transgenic non-human animal which is IFT140-deficient, it is meant a homozygous animal (i.e., the two alleles of IFT140 are defective).

Typically suitable transgenic non-human animals are laboratory animals such as rodents, rats, mice, monkeys, dogs, rabbits, guinea pigs, goats, sheep, pigs and cattle. In one embodiment the transgenic non-human animal of the present invention is a rodent. In a more preferred embodiment the transgenic animal is a mouse.

Typically the transgenic animals according to the invention are generated using General Methods Standard molecular biology techniques known in the art (see for example U.S. Pat. No. 6,740,793, US2006/0101533).

The invention provides a means for identification of agents that interfere, delay or inhibit processes involved in diseases or conditions involving a mutated IFT140 gene, such as skeletal ciliopathy. Such agents would be of significant clinical importance for treatment of skeletal ciliopathy. The provision of the animal model according to the present invention can greatly shorten the time required for screening for such agents. Thus the present invention relates to a method for identifying a compound useful for treating skeletal ciliopathy, comprising the step of contacting a candidate compound with a transgenic animal according to the invention.

The invention will further be illustrated in view of the following figures and example.

### FIGURES

FIG. 1: Pedigree of two MSS Saoudi Arabian families segregating the same IFT140 mutation and haplotype reconstructions at the 16p13.3 locus.

In family IV, first cousin parents do not share the same alleles at the D16S3024 locus in intron 3 of the IFT140 gene, supporting the existence of an intragenic recombination event. In addition, the haplotypes of their eldest unaffected daughter as well as affected individual FIII2 (Family III) suggest the occurrence of recombination events between the D16S521 and (GGA)n_{—}1,482,493 loci or between this last locus and exon 17 of the IFT140 gene. ESR: possible extragenic site of recombination; ISR: possible intragenic site of recombination; M: mutation.

FIG. 2: IFT140 gene, protein structure, and position and nature of IFT140 mutations identified in homozygote and compound heterozygote individuals affected with skeletal ciliopathies.

### EXAMPLE

The inventors collected 15 families presenting three diagnostic criteria of MSS, namely early-onset retinal dystrophy, POSE and renal disease, and 2 families with non-overt renal disease (see Table A-I to A-XVIII).

The meaning of abbreviations in Tables A is the following: CRF: chronic renal failure; D: diopters; ERG: electroretinogram; ESRD: end-stage renal disease; LP: Light perception; LRE: left and right eye; Mo: months NA: not available; POSE: phalangeal cone-shaped epiphyses; RP: retinitis pigmentosa; VA: visual acuity; VF: visual field; Yrs: years; -: no particularity.

TABLE A-I

| | Family I |
|---|---|
| Affected | I1/M |
| Individual/sex | |
| Age at | Birth, 12 yrs, 17 yrs |
| examination | |
| IFT140 allele 1 | c. 2399 + 1G > T, splice |
| IFT140 allele 2 | c. 1990G > A, p.Glu664Lys |
| Psychomotor | Normal |
| development | |
| Cerebral MRI | Normal |
| Retina | |
| Onset (discovery) | 4 months |
| Nystagmus | Yes |
| Refraction | +6.00D LRE |
| Light behavior | NA |
| VF | Severe constriction |
| VA | LP |
| Fundus | Attenuated retinal arterioles |
| ERG | Unrecordable |
| Skeletal features | |
| Stature | Short |
| Skull | No particularity |
| Thorax | Small thoracic cavity with short and thick ribs |
| Limbs | No particularity |
| Hands | PCSE |
| Hips | Areas of lucency and sclerosis in the proximal |
| | metaphyses of each femur |
| Renal features | |
| Function | CRF (12 yrs) |
| Ultrasonography | Small cystic structures (left kidney) |
| Liver | No overt hepatic dysfunction |
| Other | — |

TABLE A-II

| | Family II |
|---|---|
| Affected | II1/M |
| Individual/sex | |
| Age at examination | Birth, 6 yrs and 18 yrs |
| IFT140 allele 1 | c.932A > G, p.Tyr311Cys |
| IFT140 allele 2 | c.857-862deITTGA, p.lle286Lysfs*6 |
| Psychomotor | Normal |
| development | |
| Cerebral MRI | Normal |
| Retina | |
| Onset (discovery) | Birth |
| Nystagmus | Yes |
| Refraction | −1.25RE; −1LE |
| Light behavior | Photoattraction |
| VF | Tubular visual field |
| VA | 40/200LRE |
| Fundus | Salt and pepper aspect, macular |
| | degeneration |
| ERG | Unrecordable |
| Skeletal features | |
| Stature | Normal |
| Skull | No particularity |
| Thorax | No particularity |
| Limbs | No particularity |
| Hands | PCSE (feet) |
| Hips | No particularity |
| Renal features | |
| Function | CRF (18years), Hyperkaliemy |
| Ultrasonography | Several bilateral cysts |
| Liver | Cholestasis |
| Other | — |

TABLE A-III

| | Family III | |
|---|---|---|
| Affected | III1/F | III-2/M |
| Individual/sex | | |
| Age at | 15 yrs | 11 yrs |
| examination | | |
| IFT140 allele 1 | c. 1990G > A, p.Glu664Lys | |
| IFT140 allele 2 | c. 1990G > A, p.Glu664Lys | |
| Psychomotor | Mild ID | Mild ID (IQ = 80) |
| development | | |
| Cerebral MRI | Reported normal | Reported normal |
| Retina | | |
| Onset | Birth | 1 month |
| (discovery) | | |
| Nystagmus | Yes | Yes |
| Refraction | NA | NA |
| Light behavior | Photoattraction | Photoattraction |
| VF | No visual field | No visual field |
| VA | LP | 20/400LRE |
| Fundus | Macular degeneration | NA |
| ERG | Unrecordable | Unrecordable |
| Skeletal | | |
| features | | |
| Stature | Short (heights <5^{th} | Short (heights <5^{th} |
| | centile) | centile) |
| Skull | No particularity | Microcephaly (−2SD) |
| Thorax | No particularity | No particularity |
| Limbs | No particularity | No particularity |
| Hands | PCSE | Short, PCSE |
| Hips | Delayed ossification, | Delayed ossification, |
| | metaphyseal defect | metaphyseal defect |
| Renal features | | |
| Function | No overt renal disease | No overt renal disease |
| Ultrasonography | NA | NA |
| Liver | No overt hepatic | No overt hepatic |
| | dysfunction | dysfunction |
| Other | — | — |

TABLE A-IV

| | Family IV | | |
|---|---|---|---|
| Affected | IV3/M | IV4/F | IV5/F |
| Individual/sex | | | |
| Age at | 3 mo, 15 yrs | Birth, 4 yrs, 9 yrs | Birth, 5 yrs, 6 yrs |
| examination | and 17 yrs | | |
| IFT140 | c. 1990G > A, p.Glu664Lys | | |
| allele 1 | | | |
| IFT140 | c. 1990G > A, p.Glu664Lys | | |
| allele 2 | | | |
| Psychomot | Autistic | Autistic | Normal |
| or development | features, Seizures | features, Epilepsy | development and |
| | | Seizures | cognitive function since |
| | | | birth |
| Cerebral | Reported | Reported | NA |
| MRI | normal | normal | |
| Retina | | | |
| Onset | 3 months | Birth | Birth |
| (discovery) | | | |
| Nystagmus | Yes | Yes | Yes |
| Refraction | +7LRE | NA | NA |
| Light | Indifference | Indifference | NA |
| behavior | | | |
| VF | No visual | No visual | NA |
| | field | field | |
| VA | LP | LP | LP |
| Fundus | NA | Advanced | Diffuse pigmentary |
| | | RP aspect | changes |
| ERG | Unrecordable | Unrecordable | NA |
| Skeletal | | | |
| features | | | |
| Stature | Normal | Short | NA |
| Skull | No | Microcephaly | NA |
| | particularity | (−4SD) | |
| Thorax | No | No | NA |
| | particularity | particularity | |
| Limbs | No | No | NA |
| | particularity | particularity | |
| Hands | PCSE | Short, PCSE | NA |
| Hips | No | No | NA |
| | particularity | particularity | |
| Renal | | | |
| features | | | |
| Function | No overt | No overt | No overt renal |
| | renal dysfunction | renal dysfunction | dysfunction |
| Ultrasonography | NA | NA | NA |
| Liver | No overt | No overt | No overt hepatic |
| | hepatic dysfunction | hepatic dysfunction | dysfunction |
| Other | — | — | — |

TABLE A-V

| | Family V | |
|---|---|---|
| Affected | V1/M | V2/F |
| Individual/sex | | |
| Age at | 4 yrs, 10 yrs | 2 yrs, 4 yrs |
| examination | | |
| IFT140 | c. 634G > A, p.Gly212Arg | |
| allele 1 | | |
| IFT140 | c.3916dupp.Ala1306Glyfs*56 | |
| allele 2 | | |
| Psychomotor | Normal | Normal |
| development | | |
| Cerebral | Normal | Normal |
| MRI | | |
| Retina | | |
| Onset | 4 years | 2 years |
| (discovery) | | |
| Nystagmus | No | No |
| Refraction | Myopia | NA |
| Light | NA | NA |
| behavior | | |
| VF | Tubular | NA |
| VA | 60/200RE; 120/200 LE | 120/200RE; 100/200LE |
| | with colour vision | |
| Fundus | RP aspect | RP aspect |
| ERG | Severely altered | Severely altered |
| Skeletal | | |
| features | | |
| Stature | Short | Short(−2DS) |
| Skull | Craniostenosis, | Craniostenosis, |
| | scaphocephaly, facial | scaphocephaly, |
| | dysmorphy | microcephaly (−2SD), |
| | | facial dysmorphy |
| Thorax | NA | NA |
| Limbs | NA | NA |
| Hands | PSCE | PCSE |
| Hips | No particularity | No particularity |
| Renal | | |
| features | | |
| Function | CRF (4 yrs), ESRD | CRF (9 mo), ESRD (3 yrs) |
| | (15 yrs) | |
| Ultrasonography | Cysts, Small kidneys (−1DS); | 1 cyst (cortico-medullar, right |
| | Hyperechogenecity with | kidney); Hyperechogenecity with |
| | cortico-medullary differentiation | cortico-medullary differentiation loss; |
| | loss; Growth retardation (−2DS) | Growth retardation |
| Liver | Moderate cholestasis, | Moderate cholestasis |
| | hepatic cytolisis with | |
| | hepatomegaly, portal fibrosis | |
| Other | Bilateral hypoacousia | — |

TABLE A-VI

| | FamilyVI |
|---|---|
| Affected | VI-1/M |
| Individual/sex | |
| Age at | 2 years |
| examination | |
| IFT140 | c. 699T > G, p.Ile233Met |
| allele 1 | |
| IFT140 | c.699T > G, p.Ile233Met |
| allele 2 | |
| Psychomotor | Normal |
| development | |
| Cerebral | Normal |
| MRI | |
| Retina | |
| Onset | |
| (discovery) | |
| Nystagmus | No |
| Refraction | NA |
| Light | NA |
| behavior | |
| VF | NA |
| VA | NA |
| Fundus | Reported normal |
| ERG | Normal |
| Skeletal | |
| features | |
| Stature | Short |
| Skull | No particularity |
| Thorax | Short |
| Limbs | Short |
| Hands | Short and large PCSE, clinodactyly of the 5th finger |
| Hips | Trident |
| Renal | |
| features | |
| Function | ESRD (26 months) |
| Ultrasonography | Normal sized kidneys; Hyperechogenecity |
| | of the medulla, slim cortex; Abnormal thickening |
| | of the tubular basement membranes; |
| | Interstitial fibrosis; No cyst. |
| Liver | Hepatomegaly; hepatic fibrosisis |
| Other | — |

TABLE A-VII

| | Family VII |
|---|---|
| Affected | VI1/M |
| Individual/sex | |
| Age at examination | Birth, 8 mo, 12 mo, 18 mo |
| IFT140 allele 1 | c. 2399 + 1G > T, splice |
| IFT140 allele 2 | c. 634G > A, p.Gly212Arg |
| Psychomotor | Hypotonia and poor feeding at birth, developmental |
| development | delay |
| Cerebral MRI | Normal |
| Retina | |
| Onset (discovery) | Birth |
| Nystagmus | Yes |
| Refraction | NA |
| Light behavior | NA |
| VF | NA |
| VA | Able to fix and follow |
| Fundus | NA |
| ERG | NA |
| Skeletal features | |
| Stature | No particularity |
| Skull | No particularity |
| Thorax | Short thorax with short ribs |
| Limbs | Trident |
| Hands | PSCE |
| Hips | No particularity |
| Renal features | |
| Function | Elevated uera and proteinuria (18 mo) |
| Ultrasonography | Increased echogenicity; Non-specific |
| | tubulo-interstitial nephritis |
| Liver | NA |
| Other | — |

TABLE A-VIII

| | Family VIII |
|---|---|
| Affected individual/sex | VIII1/F |
| Age at examination | NA |
| IFT140 allele 1 | p.Gly522Glu |
| IFT140 allele 2 | no mutation |
| | identified |
| Psychomotor development | Normal |
| Cerebral MRI | NA |
| Retina | |
| Onset (discovery) | Before 1 year |
| Nystagmus | Yes |
| Fundus | NA |
| ERG | Unrecordable |
| Diagnosis | LCA |
| Skeletal features | |
| Stature | |
| Thorax | |
| Limbs | |
| Hands | PCSE |
| Hips | |
| Renal features | |
| Function | CRF (1st |
| | decade) |
| Renal biopsy and/or | NA |
| Ultrasonography | |
| Liver | No overt hepatic |
| | disease |
| Other | — |

TABLE A-IX

| | Family IX |
|---|---|
| Affected | IX1/M |
| individual/sex | |
| Age at examination | 8 mo |
| IFT140 allele 1 | p.Val292Met |
| IFT140 allele 2 | no mutation identified |
| Psychomotor | NA |
| development | |
| Cerebral MRI | NA |
| Retina | |
| Onset (discovery) | Before 1 year |
| Nystagmus | NA |
| Fundus | Thin retinal vessels, equatorial pigmentary deposits, |
| | slight macular dystrophy |
| ERG | Unrecordable |
| Diagnosis | LCA |
| Skeletal features | |
| Stature | |
| Thorax | Dystrophy |
| Limbs | |
| Hands | PCSE |
| Hips | |
| Renal features | |
| Function | Growth retardation, ESRD (8 months) |
| Renal biopsy and/or | Tubulo-interstitial nephropathy |
| Ultrasonography | |
| Liver | Hepathomegaly with elevated serum transaminase |
| | and gamma glutamyl tansferase levels |
| Other | — |

TABLE A-X

| | Family X |
|---|---|
| Affected individual/sex | X1/F |
| Age at examination | 7 yrs |
| IFT140 allele 1 | p.Arg576Gln |
| IFT140 allele 2 | no mutation identified |
| Psychomotor development | NA |
| Cerebral MRI | NA |
| Retina | |
| Onset (discovery) | Birth |
| Nystagmus | yes |
| Fundus | Dull retina, thin vessels |
| ERG | Unrecordable |
| Diagnosis | LCA |
| Skeletal features | |
| Stature | |
| Thorax | |
| Limbs | |
| Hands | PCSE |
| Hips | |
| Renal features | |
| Function | Polyuria, polydypsia, ESRD (13 yrs) |
| Renal biopsy and/or | Tubulo-interstitial nephropathy; |
| Ultrasonography | No cyst |
| Liver | No overt hepatic disease |
| Other | — |

TABLE A-XI

| | Family XI |
|---|---|
| Affected individual/sex | XI1/F |
| Age at examination | NA |
| IFT140 allele 1 | [p.Gly163Gly; p.Glu164fs10] |
| IFT140 allele 2 | no mutation |
| | identified |
| Psychomotor development | Ataxia and |
| | hypotonia |
| Cerebral MRI | Molar-tooth sign |
| Retina | |
| Onset (discovery) | First year |
| Nystagmus | yes |
| Fundus | NA |
| ERG | Unrecordable |
| Diagnosis | LCA |
| Skeletal features | |
| Stature | |
| Thorax | |
| Limbs | |
| Hands | PCSE, polydactily |
| Hips | |
| Renal features | |
| Function | ESRD (3 yrs) |
| Renal biopsy and/or | NA |
| Ultrasonography | |
| Liver | No overt hepatic |
| | disease |
| Other | — |

TABLE A-XII

| | Family XII |
|---|---|
| Affected individual/sex | XII1/M |
| Age at examination | 19 mo |
| IFT140 allele 1 | no mutation identified |
| IFT140 allele 2 | no mutation identified |
| Psychomotor development | NA |
| Cerebral MRI | NA |
| Retina | |
| Onset (discovery) | After one year |
| Nystagmus | Yes |
| Fundus | Retinitis pigmentosa aspect |
| ERG | NA |
| Diagnosis | EOSRD |
| Skeletal features | |
| Stature | Short |
| Thorax | |
| Limbs | |
| Hands | PCSE |
| Hips | Reported as abnormal |
| Renal features | |
| Function | ESRD (2 yrs) |
| Renal biopsy and/or | NA |
| Ultrasonography | |
| Liver | Fibrosis; Cytolysis |
| Other | — |

TABLE A-XIII

| | Family XIII |
|---|---|
| Affected individual/sex | XIV1/F |
| Age at examination | 18 mo |
| IFT140 allele 1 | no mutation identified |
| IFT140 allele 2 | no mutation identified |
| Psychomotor development | NA |
| Cerebral MRI | NA |
| Retina | |
| Onset (discovery) | Before 2 years |
| Nystagmus | No |
| Fundus | Retinitis pigmentosa aspect |
| ERG | NA |
| Diagnosis | EOSRD |
| Skeletal features | |
| Stature | Short |
| Thorax | |
| Limbs | |
| Hands | PCSE |
| Hips | Femoral neck dysplasia |
| Renal features | |
| Function | ESRD (6 yrs) |
| Renal biopsy and/or | Small kidneys; Dedifferentiated |
| Ultrasonography | tubules with basement membrane |
| | thickning; Arterial lesions; No cyst |
| Liver | Hepatomegaly |
| Other | — |

TABLE A-XIV

| | Family XIV |
|---|---|
| Affected | XV1/M |
| individual/sex | |
| Age at examination | 3 mo |
| IFT140 allele 1 | no mutation identified |
| IFT140 allele 2 | no mutation identified |
| Psychomotor | Normal |
| development | |
| Cerebral MRI | Normal |
| Retina | |
| Onset (discovery) | Birth |
| Nystagmus | Yes |
| Fundus | Normal aspect |
| ERG | Unrecordable |
| Diagnosis | LCA |
| Skeletal features | |
| Stature | |
| Thorax | |
| Limbs | |
| Hands | PCSE |
| Hips | |
| Renal features | |
| Function | ESRD (4 yrs) |
| Renal biopsy and/or | NA |
| Ultrasonography | |
| Liver | Cholestasis; Cysts |
| Other | — |

TABLE A-XV

| | Family XV |
|---|---|
| Affected individual/sex | XVI1/F |
| Age at examination | 4 yrs |
| IFT140 allele 1 | no mutation |
| | identified |
| IFT140 allele 2 | no mutation |
| | identified |
| Psychomotor development | Normal |
| Cerebral MRI | NA |
| Retina | |
| Onset (discovery) | NA |
| Nystagmus | NA |
| Fundus | Retinitis pigmentosa |
| | aspect |
| ERG | NA |
| Diagnosis | EOSRD |
| Skeletal features | |
| Stature | Very short |
| | (dwarfism) |
| Thorax | |
| Limbs | |
| Hands | PCSE (3rd |
| | phalange) |
| Hips | |
| Renal features | |
| Function | ESRD (5 yrs) |
| Renal biopsy and/or | |
| Ultrasonography | |
| Liver | No overt hepatic |
| | disease |
| Other | — |

TABLE A-XVII

| | Family XVII |
|---|---|
| Affected individual/sex | XVII1/F |
| Age at examination | 6 mo |
| IFT140 allele 1 | no mutation identified |
| IFT140 allele 2 | no mutation identified |
| Psychomotor development | Hypotonia |
| Cerebral MRI | Normal |
| Retina | |
| Onset (discovery) | 6 mo |
| Nystagmus | NA |
| Fundus | Retinitis pigmentosa aspect |
| ERG | Unrecordable |
| Diagnosis | LCA |
| Skeletal features | |
| Stature | Short |
| Thorax | |
| Limbs | short limbs |
| Hands | PCSE |
| Hips | |
| Renal features | |
| Function | ESRD (8 mo) |
| Renal biopsy and/or | Cysts; Normal sized kidneys; |
| Ultrasonography | Hyperechogenicity |
| Liver | Cholestasis; Cysts |
| Other | Primary cilia dyskinesia (Bronchial |
| | biopsy) |

TABLE A-XVIII

| | Family XVIII |
|---|---|
| Affected individual/sex | XVIII1/M |
| Age at examination | NA |
| IFT140 allele 1 | no mutation identified |
| IFT140 allele 2 | no mutation identified |
| Psychomotor development | NA |
| Cerebral MRI | NA |
| Retina | |
| Onset (discovery) | NA |
| Nystagmus | NA |
| Fundus | NA |
| ERG | NA |
| Diagnosis | Reported as having retinitis pigmentosa |
| Skeletal features | |
| Stature | |
| Thorax | |
| Limbs | |
| Hands | PCSE |
| Hips | |
| Renal features | |
| Function | Polyuria, polydypsia, ESRD (4 years) |
| Renal biopsy and/or | Normal sized kidneys; Hyperechogenic |
| Ultrasonography | kidneys; No cyst |
| Liver | No overt hepatic disease |
| Other | asthma |

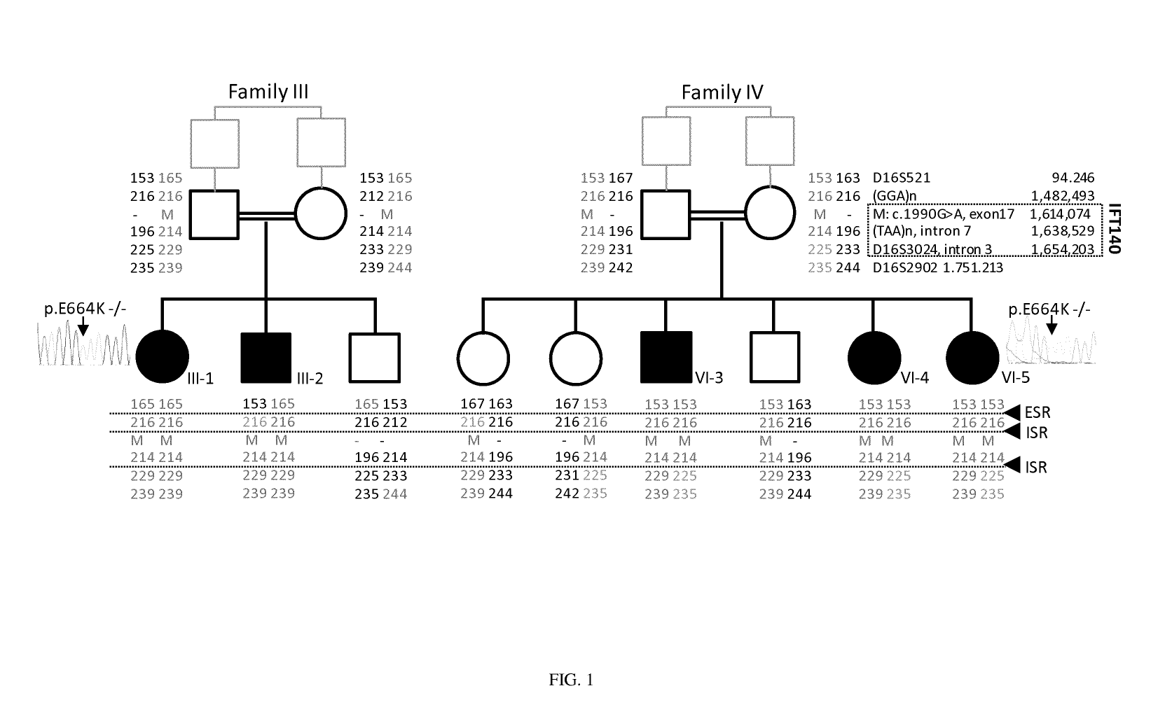

To identify the gene which mutations are responsible for MSS, the genomic DNA of an affected individual born to unrelated parents (FI1; FIG. 1 and Table A) was subjected to ciliome re-sequencing using a 5.3 Mb customized Agilent SureSelect Target Enrichment library to capture 32,146 exons of 1666 genes.

The inventors first focused our analysis on consensus splice site changes, nonsynonymous variants and insertion/deletion in coding regions.

Considering that MSS causing mutations are rare, the inventors assumed that the affected individual was likely compound heterozygote for variants absent in the dbSNP132, 1000Genome and in-house databases.

Cilia genes were compiled from the Cilia Proteome v3.0 (http://v3.ciliaproteome.org/cgi-bin/index.php) and Cildb (http://cildb.cgm.cnrs-gif.fr/) databases and by data mining of published cellular and animal models. A multiplexing approach was performed with molecular barcodes for traceable ID of samples, which were sequenced using the SOLID4 (50-bp reads) technologies (Life Technologies). For sequence alignment, variant calling and annotation, the sequences were aligned to the human genome reference sequence (hg19 assembly) using the Burrows-Wheeler Aligner (BWA) (Li, H., Durbin, R. (2010). Fast and accurate long-read alignment with Burrows-Wheeler transform. Bioinformatics 26:589-95). All calls with read coverage ≦2 and a Phred-scaled SNP quality of ≦20 were filtered out. Variants were annotated with an in-house pipeline based on the Ensembl database (release 61).

The filtering strategy reduced the number of candidate genes and one candidate gene, was pointed out: IFT140 (intraflagellar transport protein 140 homolog; [NM_{—}014714.3]), that encodes the last IFT-A component.

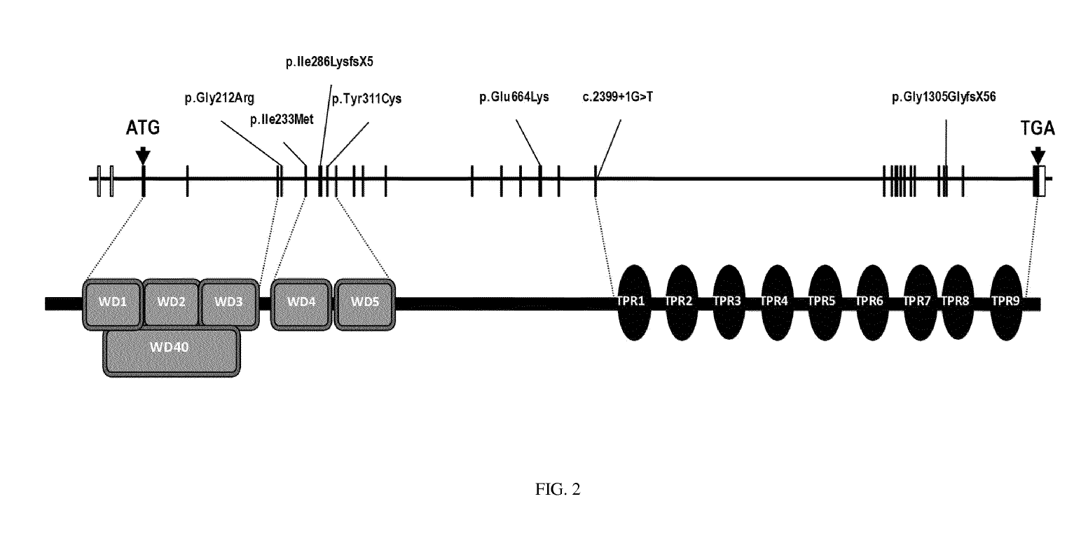

A missense and a donor splice site mutation were found (FIG. 2 and Table AI). The missense mutation altered an acidic residue conserved across species (c.1990G>A, p.Glu664Lys) whereas the splice-site mutation was expected to result in the skipping of exon 18 and/or the use of a surrounding cryptic splice site (c.2399+1G>T). Sanger sequencing confirmed these results and segregation analysis excluded allelism of the variants.

Further ciliome re-sequencing and/or Sanger sequencing of the 29 IFT140 coding exons (3 to 31, Table B) and intron-exon boundaries were done.

TABLE B
IFT140 primers used to amplify the 29 IFT140 encoding
exons and intron-exon junctions from genomic DNA of
affected individuals and their relatives.

| | Forward (5′-3′) | Reverse (5′-3′) |
|---|---|---|
| Exon 3 | Atgcttccttttcgtcattc | Caaacaacatgagccagaag |
| | (SEQ ID NO: 1) | (SEQ ID NO: 2) |
| Exon 4 | Ttcttaaaagtagctgtccctc | Ctcgagcatggtcagaaag |
| | (SEQ ID NO: 3) | (SEQ ID NO: 4) |
| Exons 5-6 | Acctgtccctccaccag | Ctaccagacacccctaaaatg |
| | (SEQ ID NO: 5) | (SEQ ID NO: 6) |
| Exon 7 | Atggaagcagataaaccagg | Agaagagaaagggccgtc |
| | (SEQ ID NO: 7) | (SEQ ID NO: 8) |
| Exon 8 | Gaggaccagagactccagg | Gagcctgttcctcaggg |
| | (SEQ ID NO: 9) | (SEQ ID NO: 10) |
| Exon 9 | agagggaaacgccactaac | Ttcactaccattattttgcagc |
| | (SEQ ID NO: 11) | (SEQ ID NO: 12) |
| Exon 10 | Gcttgttgctgttttgttc | Gtctccactttcatgcagc |
| | (SEQ ID NO: 13) | (SEQ ID NO: 14) |
| Exon 11 | Gcatctttctcctgctctg | Gctgccctgaactacctg |
| | (SEQ ID NO: 15) | (SEQ ID NO: 16) |
| Exon 12 | Cagttgcacctcacttttcc | Ctctcattaggcagggagg |
| | (SEQ ID NO: 17) | (SEQ ID NO: 18) |
| Exon 13 | Tgggaatgctcctgaaac | Caattgagaggcaggatttc |
| | (SEQ ID NO: 19) | (SEQ ID NO: 20) |
| Exon 14 | Ttcttgtgtgtacaaggttatctg | Cacctaaggagtcttctggc |
| | (SEQ ID NO: 21) | (SEQ ID NO: 22) |
| Exon 15 | Cggtgaacagaggtttcag | Gggaggacagaggtgagg |
| | (SEQ ID NO: 23) | (SEQ ID NO: 24) |
| Exon 16 | Tgtgtcagtcccctagcac | Gccgagaagtaactgaacatc |
| | (SEQ ID NO: 25) | (SEQ ID NO: 26) |
| Exon 17 | Gtgggagggagactggtag | Gatcttaagtctgtcaaatggc |
| | (SEQ ID NO: 27) | (SEQ ID NO: 28) |
| Exon 18 | Tgtgtagcaaaatggcactc | Gggctaactcacatttcagc |
| | (SEQ ID NO: 29) | (SEQ ID NO: 30) |
| Exon 19 | Taaatccagaggacgaggtc | Gaaaggaaagagccgtgac |
| | (SEQ ID NO: 31) | (SEQ ID NO: 32) |
| Exon 20 | Tgatgactacttgccccag | Tcagtgcaggctcaggc |
| | (SEQ ID NO: 33) | (SEQ ID NO: 34) |
| Exon 21 | Acagtgtgtttgagacgtgg | Aagaggcctcacaagcc |
| | (SEQ ID NO: 35) | (SEQ ID NO: 36) |
| Exon 22 | Tgatgcgtttcaccgac | Gaaagggagccgtctcag |
| | (SEQ ID NO: 37) | (SEQ ID NO: 38) |
| Exon 23- | Ggtgacatgcactctaaggg | Ttccacttgaagctctcctc |
| 24 | (SEQ ID NO: 39) | (SEQ ID NO: 40) |
| Exon 25- | Attccagaatctcagcggac | Ctctggggacaggagacc |
| 26 | (SEQ ID NO: 41) | (SEQ ID NO: 42) |
| Exon 27 | Ttcctctgaagcggtgg | Cgcagcctaactgcctg |
| | (SEQ ID NO: 43) | (SEQ ID NO: 44) |
| Exon 28- | Tgtgagagctcctgctcg | Cggccctgtagtcacatc |
| 29 | (SEQ ID NO: 45) | (SEQ ID NO: 46) |
| Exon 30 | Aggccaagagtctcctctg | Ttcgtttcaggagccttg |
| | (SEQ ID NO: 47) | (SEQ ID NO: 48) |
| Exon 31 | Gtcgtgcctggttgttc | Aaaatgctggctttgcc |
| | (SEQ ID NO: 49) | (SEQ ID NO: 50) |

Homozygote or compound heterozygote disease-causing mutations in five other MSS families (FIG. 2; Table 1, Tables A) and single heterozygote mutations in four additional ones (Table 1 and Tables A) were detected.

All changes were absent from 200 control chromosomes and were predicted to be deleterious using the Alamut Mutation Interpretation Software, a decision support system for mutation interpretation based on Align DGVD, Polyphen-2, SIFT, SpliceSiteFinder-like, MaxEntScan, NNSPLICE and Human Splicing Finder (Table 1). Segregation of compound heterozygote and homozygote mutations with the disease was confirmed.

Affected individuals FII1 and FV1/V2 were compound heterozygotes for missense and truncating mutations whereas FIII1/III2, FIV3/IV4/IV5 and FVI1 were homozygotes for missense mutations (Table 1 and FIG. 2). Affected cases FI111/III2 and FIV3/IV4/IV5 born to consanguineous parents from Saoudi Arabia harbored the c.1990G>A (p.Glu664Lys) change identified in affected individual FI1 (Table 1 and FIG. 2). Linkage analysis at the IFT140 locus detected an intragenic IFT140 recombination precluding search for linkage disequilibrium in families segregating this latter change (FIG. 1). Affected individuals FVIII1, FIX1 and FX1 were single heterozygous for missense mutations. FXI1 harbored a conservative change predicted to create 4 bp upstream of intron 5 donor splice site, a competing donor site which use would result in the apparition of a premature stop codon (c.489C>T, p.Gly163Gly/p.Glu164Thrfs*10; Table 1 and Tables A).

TABLE 1
IFT140 mutations identified in ten individuals affected with Mainzer-Saldino
Syndrome and a child with Jeune Syndrome

| | | | Allele 1 | | Allele 2 | |
|---|---|---|---|---|---|---|
| F. | Pedigree | Diag. | Mutation | Predicted effect | Mutation | Predicted effect |
| FI | Simplex, non | MSS | c.2399 + 1G > T | Loss of intron | c.1990G > A | p.Glu664Lys |
| | consanguineous | | | 19 donor splice- | | (deleterious) |
| | | | | site | | |
| FI | Simplex, non | MSS | c.2399 + 1G > T | Loss of intron | c.1990G > A | p.Glu664Lys |
| | consanguineous | | | 19 donor splice- | | (deleterious) |
| | | | | site | | |
| FII | Simplex, non | MSS | c.932A > G | p.Tyr311Cys | c.857_860de | p.Ile286Lysfs6 |
| | consanguineous | | | (deleterious) | lTTGA | |
| FIII | Multiplex, | MSS | c.1990G > A | p.Glu664Lys | c.1990G > A | p.Glu664Lys |
| | consanguineous | | | (deleterious) | | (deleterious) |
| FIV | Multiplex, | MSS | c.1990G > A | p.Glu664Lys | c.1990G > A | p.Glu664Lys |
| | consanguineous | | | (deleterious) | | (deleterious) |
| FV | Multiplex, non | MSS | c.634G > A | p.Gly212Arg | c.3916dup | p.Ala1306Glyfs56 |
| | consanguineous | | | (deleterious) | | |
| | | | | and/or alteration | | |
| | | | | exon 6 donor- | | |
| | | | | splice site | | |
| FVI | Simplex, | MSS | c.699T > G | p.Ile233Met | c.699T > G | p.Ile233Met |
| | consanguineous | | | (deleterious) | | (deleterious) |
| FVII | Simplex, non | Jeune | c.2399 + 1G > T | Loss of intron | c.634G > A | p.Gly212Arg |
| | consanguineous | syndrome | | 19 donor splice- | | (deleterious) and/or |
| | | | | site | | alteration exon 6 |
| | | | | | | donor splice site |
| FVIII | Simplex, non | MSS | c.1565G > A | p.Gly522Glu | No mutation | |
| | consanguineous | | | (deleterious) | identified | |
| FIX | Simplex, non | MSS | c.874G > A | p.Val292Met | No mutation | |
| | consanguineous | | | (deleterious) | identified | |
| FX | Simplex, non | MSS | c.1727G > A | p.Arg576Gln | No mutation | |
| | consanguineous | | | (deleterious) | identified | |
| FXI | Simplex, non | MSS | c.489C > T | p.Gly163Gly | No mutation | |
| | consanguineous | | | and creation of | identified | |
| | | | | an additional | | |
| | | | | donor splice-site | | |
| | | | | 4 bp upstream | | |
| | | | | intron 5 | | |
| | | | | predicted to | | |
| | | | | result in | | |
| | | | | c.488_491del, | | |
| | | | | p.Glu164Thrfs10) | | |

The effect of the c.634G>A (p.Gly212Arg), c.699T>G (p.Ile233Met), c.932A>G, (p.Tyr311Cys) and c.1990G>A (p.Glu664Lys) changes on the IFT140 localization was assessed in the telomerase-immortalized retinal pigment epithelial cell line (RPE1). Flagged-IFT140 mutant proteins showed a partial to nearly complete loss of basal body localization associated with an increase of cytoplasm staining while the wild-type Flagged-IFT140 protein predominantly localized to the basal bodies in RPE1 cells. The c.1990G>A (p.Glu664Lys) change displayed the most severe disorganization (IFT140 mislocalization in 80% of the cells. To assess the impact of IFT140 mutations on ciliogenesis, abundance and morphology of primary cilia were studied in cultured fibroblasts of affected individuals FII1 and FIV3. Staining of cilia axonemes using acetylated alpha-tubulin detected absent cilia in a high proportion of cells of affected cases compared to controls (mean affected cases vs mean controls: 55.10% vs 83.61%, p<0.0001), supporting a defect in ciliogenesis and/or cilia maintenance.

To determine the effect of the IFT140 mutations on retrograde intraflagellar transport, the inventors analyzed the endogenous subcellular localization of IFT140. The fibroblasts of affected individuals FII1 (compound heterozygote for a splice-site mutation and the c.932A>G, p.Tyr311Cys change) and FIV3 (homozygote for the c.1990G>A, p.Glu664Lys change) exhibited an unaltered IFT140 localization along the cilia axoneme. However, two components of the anterograde transport IFT-B complex, IFT88 and IFT46, were evenly distributed along the cilium of both affected individual fibroblasts whereas they were predominantly detected at the base and the tip of the cilium in control fibroblasts (p<0.0001), suggesting an alteration in retrograde ciliary transport.

The defect in ciliogenesis and/or ciliary maintenance, and the aberrant distribution of IFT88 and IFT46 in cells of affected individuals is consistent with the report of short cilia and aberrant distribution of IFT88 in mutant rempA, the drosophila homolog of IFT140¹³. The ternary IFT-B subcomplex IFT52/IFT88/IFT46 is crucial for the stabilization of the IFT-B particule¹⁴⁻¹⁶. Therefore, the data the inventors report further support the view that alterations of IFT-A components disorganize assembly and/or maintenance of ciliary structure by impairing retrograde IFT with redistribution of ciliary proteins (notably IFT-B complex components)^{5, 7, 8, 17-18}.

Genetic and clinical heterogeneity are hallmarks of multisystemic ciliopathies¹⁻². Besides, several examples have been reported which demonstrate that different mutations in a same gene, e.g. WDR35^{6,11}, WDR19 [MIM608151]⁸, CEP290 [MIM610142]¹⁹ IQCB1 [MIM609237]²⁰⁻²² can give rise to a broad range of phenotypes from isolated nephronophtisis or LCA to multisystemic, and sometimes embryonically lethal conditions. Genotype-phenotype correlations and global mutation load in ciliary genes have been suggested to account for this clinical variability^{1,2}. IFT140 mutations consistently caused phalangeal cone-shaped epiphyses with retinal dystrophy and occasional chronic renal failure, hepatic fibrosis, additional skeletal abnormalities or neurological symptoms.

The Tables A-I to AVII above disclose the clinical features of affected individuals harbouring compound heterozygous or homozygous IFT140 mutations.

The clinical clinical features of individuals are consistent with the diagnosis of MSS with the exception of FVII1 whose phenotype is consistent with Jeune syndrome.

The Tables AVIII-A-XVII above disclose Clinical features of MSS individuals with one or no IFT140 mutation.

Interestingly, while retinal dystrophy is an occasional feature of skeletal ciliopathies¹⁻² it is very uncommon in patients with IFT-A mutations^{5-8 and 10}. Conversely, all affected individuals with IFT140 mutations and full ophthalmological examination (n=9/10; 6 families) had LCA or early-onset severe retinal dystrophy between birth and 4 years of age (Tables A). With regard to individual FVI1 who had no reported retinal disease at the age of 2 years, electrophysiological recordings were not available to assess the function of photoreceptors which alteration typically precedes fundus changes. Retinal dystrophy appears therefore to be a very stringent clinical manifestation of IFT140 alterations. Although there is no clear-cut correlation between tissue expression of IFTs and clinical features the recent report of a role of IFT140 in photoreceptor cell ciliogenesis²⁴ is consistent with the retinal involvement in affected individuals with IFT140 disease alleles.

Chronic renal failure is an inclusion criterion in MSS. However, age of onset and outcome of the dysfunction vary, even within families¹². Therefore, it is possible that affected individuals from families III and IV (n=5, age-range 4-17 years) express renal failure late in the course of the disease. This would be consistent with the recent report in mouse of pronounced renal cystic disease resulting from HoxB7-Cre-driven loss of ift140 in renal collecting duct²³. Nevertheless, affected individuals from families III and IV were homozygous for a missense mutation (c.1990G>A, p.Glu664Lys) while all affected persons with renal failure but one (FVI1) harbored a severe truncating mutation (in addition to a missense mutation), raising the question to know whether some genotype-phenotype correlations may exist. From this point of view, it is worth noting that affected individual FVI1 harbored a homozygous missense mutation (c.699T>G, p.Ile233Met) and heterozygous mutations in other ciliopathy genes, TMEM67 ([MIM 609884], data not shown) and XPNPEP3 ([MIM 613553]) which could contribute to kidney failure.

Cerebral MRIs were consistently normal but sibs of two families presented with neurological symptoms including mild intellectual symptoms or autistic features associated with seizures and epilepsy (Families III and IV; Tables A). Considering the high degree of consanguinity of the two families, up to date it is difficult to decide whether some or all these traits are accounted for by IFT140 mutations or if these disabilities are independently inherited. Some of the skeletal abnormalities of individuals with IFT140 mutations overlap with clinical symptoms of Jeune and Sensenbrenner syndromes namely short hands, hip and cranial abnormalities, narrow chest and/or short stature (Tables A). This overlap opens the debate whether some affected individuals with IFT140 mutations are affected with complex MSS, or incomplete Jeune or Sensenbrenner syndromes, (e.g. FI1 who had narrow chest but no respiratory deficiency and no trident acetabular roof or FVI1 who had trident acetabular roof, a narrow chest and short ribs; Table A). Beyond, this clinical overlap raises the question to know whether other skeletal ciliopathies are accounted for by IFT140 alterations. So far the screening of large cohorts of individuals affected with Sensenbrenner of Jeune syndromes failed to detect IFT140 mutations⁸. However, during this study, the inventors had the opportunity to identify IFT140 mutations in a child affected with Jeune syndrome (FVII1, Table S1). The child was compound heterozygous for a splice-site mutation and the c.634G>A (p.Gly212Arg) change identified in Family V. These data suggest that mutations in IFT140 may be a rare cause of Jeune syndrome and that there is no clear correlation between the IFT140 genotype and the severity of the disease.

The clinical presentation of the disease did not differ significantly in MSS individuals with two IFT140 disease alleles compared to single heterozygote patients (4/17) or affected individuals with no mutation (7/18; Tables A). It is possible that some of the latter 10/17 affected individuals may harbor undetected IFT140 mutations lying in unscreened regions such as UTRs or introns. Beyond, following the example of the deep intronic CEP290 c.1991+1655A>G mutation which account for approximately 60% of CEP290 disease alleles in LCA, it is possible that a common undetected IFT140 mutation may exist19. However, like most other ciliopathies, MSS may be genetically heterogeneous with some or all 10/17 affected individuals, including single heterozygotes, harboring mutations in other genes.

In summary, the inventors here report on compound heterozygosity or homozygosity for IFT140 mutations in six MSS families and an individual affected with Jeune syndrome.

### WEB RESOURCES

The URLs for data presented herein are as follows:

Align DGVD, Polyphen-2, SIFT, SpliceSiteFinder-like, MaxEntScan, NNSPLICE and Human Splicing Finder available through the Alamut Interpretation Software 2.0, http://alamut.interactive-biosoftware.com Genome browser, http://genome.ucsc.edu Online Mendelian Inheritance in Man (OMIM), http://www.omim.org

### REFERENCES

Throughout this application, various references describe the state of the art to which this invention pertains. The disclosures of these references are hereby incorporated by reference into the present disclosure.
1. F. Hildebrandt, T. Benzing and N. Katsanis. Ciliopathies. N. Engl. J. Med., 364 (2011), pp. 1533-1543.
2. A. M. Waters and P. L. Beals. Ciliopathies: an expanding disease spectrum. Pediatr. Nephrol., 26 (2011), pp. 1039-1056.
3. D. G. Cole and W. J. Snell SnapShot: Intraflagellar transport. Cell, 137 (2009), p.784-el.
4. P. L. Beales, E. Bland, J. L. Tobin, C. Bacchelli, B. Tuysuz, J. Hill, S. Rix, C. G. Pearson, M. Kai, J. Hartley et al. IFT80, which encodes a conserved intraflagellar transport protein, is mutated in Jeune asphyxiating thoracic dystrophy. Nat. Genet., 39 (2007), pp. 727-729.
5. J. Walczak-Sztulpa, J. Eggenschwiler, D. Osborn, D. A. Brown, F. Emma, C. Klingenberg, R. C. Hennekam, G. Torre, M. Garshasbi, A. Tzschach et al. Cranioectodermal Dysplasia, Sensenbrenner syndrome, is a ciliopathy caused by mutations in the IFT122 gene. Am. J. Hum. Genet., 86 (2010), pp. 949-956.
6. C. Gilissen, H. H Arts, A. Hoischen, L. Spruijt, D. A Mans, P. Arts, B. van Lier, M. Steehouwer, J. van Reeuwijk, S. G. Kant et al. Exome sequencing identifies WDR35 variants involved in Sensenbrenner syndrome. Am. J. Hum. Genet., 87 (2010), pp. 418-423.
7. H. H. Arts, E. M. Bongers, D. A. Mans, S. E. van Beersum, M. M. Oud, E. Bolat, L. Spruijt, E. A. Cornelissen, J. H. Schuurs-Hoeijmakers, N. de Leeuw, et al. C140RF179 encoding IFT43 is mutated in Sensenbrenner syndrome. J. Med. Genet., 48 (2011), pp. 390-395.
8. C. Bredrup, S. Saunier, M. M. Oud, T. Fiskerstrand, A. Hoischen, D. Brackman, S. M. Leh, M. Midtbo, E. Filhol, C. Bole-Feysot et al. Ciliopathies with skeletal anomalies and renal insufficiency due to mutations in the IFT-A gene WDR19. Am. J. Hum. Genet., 89 (2011), pp. 634-643.
9. D. P. Cavalcanti, C. Huber, K. H. Sang, G. Baujat, F. Collins, A. L. Delezoide, N. Dagoneau, M. Le Merrer, J. Martinovic, M. F. Mello et al. Mutation in IFT80 in a fetus with the phenotype of Verma-Naumoff provides molecular evidence for Jeune-Verma-Naumoff dysplasia spectrum. J. Med. Genet., 48 (2011), pp. 88-92.
10. E. E Davis, Q. Zhang, Q. Liu, B. H. Diplas, L. M. Davey, J. Hartley, C. Stoetzel, K. Szymanska, G. Ramaswami, C. V. Logan et al. NISC Comparative Sequencing Program. TTC21 B contributes both causal and modifying alleles across the ciliopathy spectrum. Nat. Genet., 43 (2011), 189-196.
11. P. Mill, P. J. Lockhart, E. Fitzpatrick, H. S. Mountford, E. A. Hall, M. A. Reijns, M. Keighren, M. Bahlo, C. J. Bromhead, P. Budd et al. Human and mouse mutations in WDR35 cause short-rib polydactyly syndromes due to abnormal ciliogenesis. Am. J. Hum. Genet., 88 (2011), pp. 508-515.
12. R. K. Beals and R. G. Weleber. Conorenal dysplasia: a syndrome of cone-shaped epiphysis, renal disease in childhood, retinitis pigmentosa and abnormality of the proximal femur. Am. J. Med. Genet., 143A (2007), pp. 2444-2447.
13. E. Lee, E. Sivan-Loukianov, D. F. Eberl and M. J. Kernan. An IFT-A protein is required to delimit functionally distinct zones in mechanosensory cilia. Curr. Biol., 18 (2008), pp. 1899-1906.
14. B. F. Lucker, M. S. Miller, S. A. Dziedzic, P. T. Blackmarr and D. G. Cole, D. G. Direct Interactions of Intraflagellar Transport Complex B Proteins IFT88, IFT52, and IFT46. J. Biol. Chem., 285 (2010); pp. 21508-21518.
15. Hou Y., Qin H., Follit J. A., Pazour G. J., Rosenbaum J. L. and Witman G. B. Functional analysis of an individual IFT protein: IFT46 is required for transport of outer dynein arms into flagella. J. Cell Biol., 176 (2007), pp. 653-665.
16. N. T. Ahmed, C. Gao, B. F. Lucker, D. G Cole and D. R. Mitchell. ODA16 aids axonemal outer row dynein assembly through an interaction with the intraflagellar transport machinery. J. Cell Biol., 183 (2008), pp. 313-322.
17. S. Absalon, T. Blisnick, L. Kohl, G. Toutirais, G. Dore, D. Julkowska, A. Tavenet and P. Bastin. Intraflagellar transport and functional analysis of genes required for flagellum formation in trypanosomes. Mol. Biol. Cell., 19 (2008), pp. 929-944.
18. C. lomini, L. Li, J. M. Esparza and S. K. Dutcher. Retrograde intraflagellar transport mutants identify complex A proteins with multiple genetic interactions in Chlamydomonas reinhardtii. Genetics, 183 (2009), pp. 885-896.
19. F. Coppieters, S. Lefever, B. P. Leroy, E. De Baere. CEP290, a gene with many faces: mutation overview and presentation of CEP290base. Hum. Mutat., 31 (2010), pp. 1097-1108. Review.
20. E. A. Otto, B. Loeys, H. Khanna, J. Hellemans, R. Sudbrak, S. Fan, U. Muerb, J. F. O'Toole, J. Helou, M. Attanasio et al. Nephrocystin-5, a ciliary IQ domain protein, is mutated in Senior-Loken syndrome and interacts with RPGR and calmodulin. Nat. Genet., 37 (2005), pp. 282-288.
21. A. Estrada-Cuzcano, R. K. Koenekoop, F. Coppieters, S. Kohl, I. Lopez, R W Collin, E. B. De Baere, D. Roeleveld, J. Marek, A. Bernd et al. IQCB1 mutations in patients with leber congenital amaurosis. Invest. Ophthalmol. Vis. Sci., 52 (2011), pp. 834-839.
22. E. M. Stone, A. V. Cideciyan, T. S. Aleman, T. E. Scheetz, A. Sumaroka, M. A. Ehlinger, S. B. Schwartz, G. A. Fishman, E. I. Traboulsi, B. L. Lam et al. Variations in NPHP5 in patients with nonsyndromic leber congenital amaurosis and Senior-Loken syndrome. Arch. Ophthalmol., 129 (2011), pp. 81-87.
23. J. A. Jonassen, J. Sanagustin, S. P. Baker and G. J. Pazour. Disruption of IFT Complex A causes cystic kidneys without mitotic spindle misorientation. J. Am. Soc. Nephroi., (2012) Jan. 26.
24. T. Sedmak, U. Wolfrum. Intraflagellar transport proteins in ciliogenesis of photoreceptor cells. Biol. Cell., 103 (2011), pp 449-466.
25. Li, H., Durbin, R. (2010). Fast and accurate long-read alignment with Burrows-Wheeler transform. Bioinformatics 26:589-95.
26. Dunnen J T, Antonarakis; Mutation nomenclature extensions and suggestions to describe complex mutations: a discussion. SE. Hum Mutat. 2000 15(1):7-12.

1. A method for diagnosing a skeletal ciliopathy in a subject, said method comprising detecting a mutation in the IFT140 gene in a biological sample obtained from said subject, wherein the presence of an IFT140 mutation is indicative of a skeletal ciliopathy or a risk of having a skeletal ciliopathy.

2. The method according to claim 1 wherein the IFT140 mutation is homozygous.

3. The method according to claim 1 wherein the subject has a retinal dystrophy.

4. A method for predicting a risk of a subject to transmit a skeletal ciliopathy to his infants, said method comprising detecting a mutation in the IFT140 gene in a biological sample obtained from said subject, wherein the presence of an IFT140 mutation is indicative of a risk of transmitting a skeletal ciliopathy.

5. The method according to claim 1 wherein the skeletal ciliopathy is a Mainzer Saldino Syndrome or a Jeune Syndrome.

6. The method according to claim 1 wherein the skeletal ciliopathy is a Mainzer Saldino Syndrome.

7. A method for detecting a subject carrying a defective IFT140 gene, which method comprises detecting a mutation in the IFT140 gene in a biological sample obtained from said subject.

8. The method according to claim 1 wherein the IFT140 mutation results in a defect in ciliogenesis and/or cilia maintenance.

9. The method according to claim 1 wherein the IFT140 mutation is a missense mutation, a donor splice mutation or a truncating mutation.

10. The method according to claim 1 wherein the IFT140 mutation is selected from the group consisting of c.2399+1G>T, c.932A>G, c.1990G>A, c.634G>A, c.699T>G, c.1565G>A, c.874G>A, c.1727G>A, c.489C>T, c.857_{—}860delTTGA and c.3916dup.

11. (canceled)

12. A method for treating a skeletal ciliopathy with comprises the step of administering a IFT140-encoding polynucleotide to a patient in need thereof.

13. The method according to claim 12 wherein the skeletal ciliopathy is a Mainzer Saldino Syndrome.

14. A transgenic non-human animal which is IFT140-deficient.
